(19) United States
(12) Patent Application Publication
(10) Pub. No.: US 2007/0088228 A1
(43) Pub. Date: Apr. 19, 2007

(54) Metabolic rate measuring apparatus

(76) Inventors: Tsuyoshi Uchida, Tokyo (JP); Hiroshi Mitsumaki, Tokyo (JP); Masashi Kiguchi, Kawagoe (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/319,166

(22) Filed: Dec. 28, 2005

(30) Foreign Application Priority Data
Oct. 14, 2005 (JP) 2005-300797

(51) Int. Cl. A61B 5/00

(52) U.S. Cl. 600/549

(57) ABSTRACT

A metabolic rate measuring apparatus, which calculates metabolic rate based on the physiological parameters measurements. The apparatus measures the physiological parameters, and uses equations of metabolic rate calculates in consideration with thermal equilibrium of human body to facilitate the measurement of metabolic rate.

### CLAIM OF PRIORITY

The present application claims priority from Japanese application JP 2005-300797 filed on Oct. 14, 2005, the content of which is hereby incorporated by reference into this application.

### FIELD OF THE INVENTION

The present invention is directed to a metabolic rate measuring apparatus, which measures a plurality of physiological parameters without blood sampling, and then calculates a metabolic rate based on the plurality of physiological parameters.

### BACKGROUND OF THE INVENTION

There have been several methods for measuring the metabolic rate. Indirect calorimetric methods measure the amount of oxygen consumed in order to produce the metabolism, instead of directly measuring the metabolic rate itself. Among indirect calorimetry there is an open circuit indirect calorimetric method, which measures the amount of consumed oxygen and carbon dioxide, by putting a mask on the subject face and by collecting the breathed air. This method is used for measuring the variation of metabolic rate in course of physical exercise and during rest. As wearing a mask for a long period of time is difficult, this method is applied for the metabolic rate measurement of a short period of time.

For measuring the metabolic rate for a longer time, a closed circuit indirect calorimetric metabolic rate measurement method is used, which put the subject in a small sealed chamber to measure the consumption rate of oxygen and production rate of carbon dioxide therein. This method is applicable to the metabolic rate measurement during rest and sleep, which method is fewer burdens to the subject, suitable for the measurement of longer period. There is also another method in which the subject drinks water including stable isotopes of hydrogen and oxygen harmless to the human body, then the process to excreting the stable isotope in the urine is measured to measure the energy consumption from the intake to the excretion.

This measurement method measures the mean energy consumption rate in the quotidian life of the subject. In the JP-A No. 329542/2004 discloses a method for calculating the blood sugar value by means of the relationship between the blood sugar level and parameters corresponding to a plurality of temperature measurements derived from the body surface and the blood oxygen level.

### SUMMARY OF THE INVENTION

So far the metabolic rate measurement requires a large apparatus, which is not easily applicable to daily personal use by an individual.

The present invention has its primary object to provide a method and apparatus, which facilitate and simplify the metabolic rate measurement by an individual at home.

Most calorimetric energy produced by the metabolism is consumed for the temperature homeostasis, and the redundant heat energy is released to the outside of human body. In other words this means that the calorimetric energy produced by the metabolism (thermogenesis) is equal to the sum of the calorimetric energy stored within the body (heat reserve) and the heat released to outside (radiation). This heat control mechanism of human body may be used for the calculation of metabolic rate based on the heat reserve and the heat radiation.

The metabolic rate measuring apparatus provided according to the present invention incorporates, an environmental thermometric sensor unit for measuring environmental temperature; a first position thermometric sensor unit for measuring temperature derived from a first position on body surface of a subject; a second position temperature acquiring mean acquiring temperature of a second position, the second position being different from said first position at the subject; a blood flow information acquiring mean acquiring information about the blood flow at said first position; a storage unit for storing the environment temperature, the temperature derived from said first position, the temperature of said second position, and relationship between metabolic rate and the blood flow; a processing unit for computing the metabolic rate by applying the environment temperature measured by said environment thermometric sensor unit, first position temperature measured by said first position thermometric sensor unit, second position temperature acquired by said second position temperature acquiring mean, and the blood flow information acquired by said blood flow information acquiring mean to the relationship stored in said storage unit; and a display unit for displaying the computational result from said processing unit.

The metabolic rate measurement apparatus according to the present invention further incorporates, an environment temperature sensor unit for measuring the environmental temperature; a body surface contact unit for contacting with a first position of the body surface, a cylinder member, contacted to said body surface contact unit and having an opening at one end; a radiation thermometric sensor mounted in the vicinity of the other end of said cylinder member, for measuring radiation heat from said first position; a blood flow information acquiring mean acquiring information about the blood flow at said first position; a light source for emitting light of at least two different wavelengths into the one end of said cylinder member; a light sensor unit for detecting light interacted with said body surface; a second position temperature acquiring mean acquiring temperature of a second position, the second position being different from said first position at said body surface; a processing unit for computing the metabolic rate by applying the environment temperature measured by said environment thermometric sensor unit, temperature of first position measured by said radiation thermometric sensor unit, temperature of second position acquired by said second position temperature acquiring mean, and blood flow rate information acquired by said blood flow information acquiring mean, and light detection result detected by said light detector unit to previously stored equation of the metabolic rate; and a display unit for displaying output from said processing unit.

The a second position temperature acquiring mean may be a thermometer so as to supply the output from the thermometer to the processing unit. The a second position temperature acquiring mean may also have an operation unit for inputting the body temperature as numerical data. The first position thermometric sensor unit may also be the one having a pad unit for placing a finger, and a temperature sensor for measuring the temperature derived from the first position of the finger placed on the pad unit.

According to the present invention, a simple metabolic rate measurement apparatus can be achieved.

### BRIEF DESCRIPTION OF DRAWINGS

FIG. 1 shows a schematic block diagram indicative of the relationship between the measurements from various sensors and the parameter derived therefrom;

FIG. 2 shows a top plan view of metabolimeter according to the present invention;

FIG. 3 shows a top plan view of metabolimeter according to the present invention;

FIG. 4 shows a manual input operation of numerical values;

FIG. 5 shows the operation procedure of the apparatus;

FIGS. 6A, 6B, and 6C show a detailed block diagram of a measuring unit;

FIG. 7 shows a schematic block diagram indicative of the flow of data processing within the apparatus according to the present invention;

FIG. 8 shows a schematic block diagram indicative of the location of data storage within the apparatus according to the present invention;

FIG. 9 shows a plot of the calculation value of metabolic rate according to the present invention vs. the metabolic rate measured by the Prior Art; and

FIG. 10 shows a plot of the calculated values of glucose concentration according to the present invention vs. measurements of glucose concentration by an enzymatic electrode method.

### DETAILED DESCRIPTION OF THE PREFERRED EMBODIMENTS

Based on the principle of human body heat control mechanism, since the calorimetric energy produced by the metabolism (thermogenesis) is equal to the sum of the calorimetric energy stored within the body (heat reserve) and the heat released to outside (radiation), the following equation may be given:
(metabolic rate of whole body [thermogenesis])=(heat reserve of whole body)+(heat radiation from whole body) (1)

By defining T as the temperature in the body at the position rᵢ of the body, T_{T} as the tissue temperature at the body position rᵢ, and αI as the heat capacity at the body position ri, the heat reserve of the whole body may be the sum of the reserve at each part of the body, and can be given by the following equation:
Heat reserve of the whole body=Σᵢαᵢ{T(rᵢ)−T_{T}(rᵢ)} (2)

Equation 2 designates the amount of heat at each part of the body, and here the arterial blood temperature at a body depth is used as the effective value representative of the internal body temperature, and the temperature Tc as the effective value representative of the body tissue temperature. The heat capacity can be represented by α as the effective value representative of the heat capacity of the whole body. The term α may be a value depending on the composition, density, and body weight of human body of each individual. From the foregoing the heat reserve of the whole body can be given by the following equation:
Heat reserve of whole body=α(Ta−Tc) (3)

The heat radiation in general is through the conduction, radiation, convection, and evaporation. The conduction and radiation may have small amount of calorimetric heat when an individual is wearing clothes and can be neglected. The evaporation may also be neglected, provided that the temperature is controlled without perspiration in a room. When considering therefore the heat radiation by the convection between the body surface and the surrounding air, The heat radiation of the whole body may be the sum of the heat radiation from the body surface, and by defining the skin temperature at the body surface point rⱼ as Tₛ, external temperature in contact with the body surface point rⱼ as T_{OUT}, convection heat conduction rate at the body surface point rⱼ as βⱼ, the following equation can be given:
Heat radiation of the whole body=Σⱼβⱼ{T_{S}(rⱼ)−T_{OUT}(rⱼ)} (4)

Equation 4 considers the heat amount at each part of body, where defining the skin temperature of the finger T_{FS} as the effective value representative of the body surface temperature, the room temperature T_{R} as the effective value representative of the external temperature. For the convection heat conduction rate, β is defined as the effective value representative of the convection heat conduction rate of the entire surface area of the whole body. This β is a value depending on the surface area and worn clothes of each individual. The heat radiation from the whole body may be given by the following equation:
Heat radiation from whole body=β(T_{FS}−T_{R}) (5)

In this preferred embodiment, the reason why a finger is used for a part representative of the heat radiation from the whole body is that the finger is sensitive to the change in the ambient temperature, expresses sufficiently largely the difference of skin temperature and radiation, as well as has a small local heat production due to less muscle to reflect the change in the whole body.

The metabolic rate of whole body may be given from the equations (1), (3), and (5) as following equation:
Metabolic rate of whole body (thermogenesis)=α(Ta−Tc)+β(T_{FS}−T_{R}) (6)

From the conservation law of heat flow in the finger, the incoming heat to the finger and the outgoing heat from the finger can be thought to be in equilibrium. For the finger tissue, Now defining the arterial temperature in depth of the finger as T_{Fa}, the heat conduction rate of the finger tissue as 1, blood flow rate in the finger tissue as ω_{b}, the specific heat of blood as c_{b}, convection heat conduction rate of the finger surface as κ, the conservation law of heat flow in the finger can be given by the following equation, where χ designates to a factor of proportionality.
λ(χω_{b}c_{b}T_{Fa}−T_{FS})=κ(T_{FS}−T_{R}) (7)

The left part indicates the heat displacement from the artery to the tissue at the finger surface, which is also based on the living tissue heat displacement equation by Penn (Pennes H. H., “Analysis of tissue and arterial blood temperatures in the resting human forearm” J. applied physiology, 1, 93-122(1948)).

Here assuming that the artery temperature in depth of the finger TFa is in relation to the arterial blood temperature Ta, and T_{Fa}=σTa+τ, then the equation (7) may be written as follows:

$${T}_{a} = \frac{1}{σχ {c}_{b}} \{ \frac{κ}{λ} ( {T}_{FS} - {T}_{R} ) / {ω}_{b} + {T}_{FS} / {ω}_{b} \} - \frac{τ}{σ} \qquad ( 8 )$$

By substituting equation (8) with equation (6), equation (9) can be yielded.
(Metabolic rate of whole body [thermogenesis])

$$= \frac{α}{σχ {c}_{b}} \{ \frac{κ}{λ} ( {T}_{FS} - {T}_{R} ) / {ω}_{b} + {T}_{FS} / {ω}_{b} \} + β ( {T}_{FS} - {T}_{R} ) - α {T}_{c} - \frac{α τ}{σ} \\ = a ( {T}_{FS} - {T}_{R} ) / {ω}_{b} + {bT}_{FS} / {ω}_{b} + c ( {T}_{FS} - {T}_{R} ) + {dT}_{c} + e \\ a = \frac{α}{σχ {c}_{b}} \frac{κ}{λ} , b = \frac{α}{σχ {c}_{b}} , c = β , d = - α , e = - \frac{ατ}{σ} \qquad ( 9 )$$

In order to user equation (9), the relational factors a to e must be determined, which differ from person to person. To do this, the metabolic rate of an individual is measured to conduct a multiple regression analysis of the factor of each individual based on equation (9) and the metabolic rate measurements to derive the metabolic rate.

According to the modeled equations as above, The metabolic rate can be determined by measuring the temperature of finger surface T_{FS} as the effective value representative of the body surface temperature, the room temperature T_{R}, The body temperature Tc as the effective value representative of the tissue temperature of the body, and the blood flow rate ω_{b} then by using the proportional factors measured separately. It should be noted here that although the finger is described as a typical measuring point in the body, any other part of the body surface is equally available. Some exemplary measurement points of the body tissue temperature may be selected from such as the sublingual, infra-axillary, or rectum temperature, such that the temperature is measured on the points different from the body temperature measurement positions as effective values representative of the body surface temperature, in order to obtain the temperature corresponding to the body tissue temperature. The temperature measurement locations used as effective value representative of the body surface temperature may also include, for example, one of the limbs instead of the finger surface.

Since the measurement of metabolic rate requires a massive apparatus, the metabolic rate may be determined which corresponds to the blood glucose concentration by means of the relationship between the blood glucose concentration that is the source of energy, and the metabolic rate. The relationship between the metabolic rate of whole body and the glucose concentration may be given by the following equation, by defining the calorimetric value produced by the glucose calorigenic production at the body part rk as QG(rj), and the calorimetric value produced by the heat production from any substance other than the glucose such as the lipids and aminoic acids as QΠ(rⱼ), thus the metabolic rate of the whole body is the calorimetric sum produced on each body part:
(Metabolic rate of whole body [thermogenesis])=Σₖ{QG(rₖ)+QΠ(rₖ)} (10)

Equation (10) considers the calorimetric value of each body part, however, when assuming that the mean value of intracellular glucose concentration Gc of the whole body is in proportion to the metabolic rate by the intracellular glucose, the mean intracellular concentration Π of a substance other than the glucose in the whole body is in proportion to the metabolic rate by the substrate other than the intracellular glucose, and the factors of proportion is designated as A and B, the metabolic rate of the whole body may be described by the following equation:
Metabolic rate of whole body=AGc+BΠ (11)

From equations (9) and (11), the following equation can be derived:
AG_{c}=a(T_{FS}−T_{R})/ω_{b}+bT_{FS}/ω_{b}+c(T_{FS}−T_{R})+dT_{c}+e−BΠ (12)

According to the foregoing modeled equations, by measuring the finger surface temperature T_{FS} as the effective value representative of body surface temperature, the room temperature T_{R}, the body temperature Tc as the effective value representative of the body tissue temperature, and the blood flow rate ω_{b} and then by applying the proportional factors measured separately, the metabolic rate AGc corresponding to the blood glucose can be measured.

To improve the precision, consider the tissue oxygen saturation. The heat radiation of the whole body is in proportion to the amount of used oxygen, thus a proportional expression Gc+Π∝[O₂ consumption]. Tissue oxygen saturation StO₂ means the O₂ consumption, therefore can be given by the following expression:
StO₂=a′Gc+b′Π (13)

From the equations (12) and (13), the following equation (14) can be yielded.

$$( \frac{{Ab}^{′} - {a}^{′} B}{{b}^{′}} ) {G}_{c} = a ( {T}_{FS } - {T}_{R} ) / {ω}_{b} + {bT}_{FS} / {ω}_{b} + c ( {T}_{FS} - {T}_{R} ) + {dT}_{c} + e - \frac{B}{{b}^{′}} {St O}_{2} \qquad ( 14 )$$

According to the foregoing modeled equation, by measuring the finger surface temperature T_{FS} as the effective value representative of body surface temperature, the room temperature T_{R}, the body temperature Tc as the effective value representative of the body tissue temperature, and the blood flow rate ω_{b}, and the tissue oxygen saturation StO₂, then by applying the proportional factors measured separately, ((ab′−a′b)/b′)Gc which is the metabolic rate corresponding to the blood glucose, can be measured.

Equation (15) can be yielded by rewriting the factors in equation (14), to calculate the glucose concentration.

$${G}_{c} = a ( {T}_{FS} - {T}_{R} ) / {ω}_{b} + {bT}_{FS} / {ω}_{b} + c ( {T}_{FS} - {T}_{R} ) + {dT}_{c} + {eSt O}_{2} + f \\ a = ( \frac{{b}^{′}}{{ab}^{′} - {a}^{′} b} ) \frac{α}{σχ {c}_{b}} \frac{κ}{λ} , b = ( \frac{{b}^{′}}{{ab}^{′} - {a}^{′} b} ) \frac{α}{σχ {c}_{b}} \\ c = ( \frac{{b}^{′}}{{ab}^{′} - {a}^{′} b} ) β , d = - ( \frac{{b}^{′}}{{ab}^{′} - {a}^{′} b} ) α \\ e = - ( \frac{{b}^{′}}{{ab}^{′} - {a}^{′} b} ) \frac{b}{{b}^{′}} , f = - ( \frac{{b}^{′}}{{ab}^{′} - {a}^{′} b} ) \frac{ατ}{σ} \qquad ( 15 )$$

Now the preferred embodiment of the present invention will be described in greater details with reference to the accompanying drawings herein below.

First, the finger surface temperature and the room temperature may be readily measured if the thermometric resistance or the radiation thermometer for measuring the radiation heat is used for example. The body temperature may be measured with an electronic thermometer equipped with the thermometric resistor or an auditory meatus thermometer using the radiation thermometer may be relatively easily used to measure the sublingual, axilla, rectum, or tympanic membrane temperature. The blood flow rate may be measured with a laser blood flow meter using for example the Doppler effect. The body temperature can also be measured by adding a minute amount of heat to a finger to evaluate the temperature change. The tissue oxygen saturation can be measured uninvasively with a pulse oxymeter or tissue oxygen saturation meter equipped with a feature for determining optically oxygen saturation.

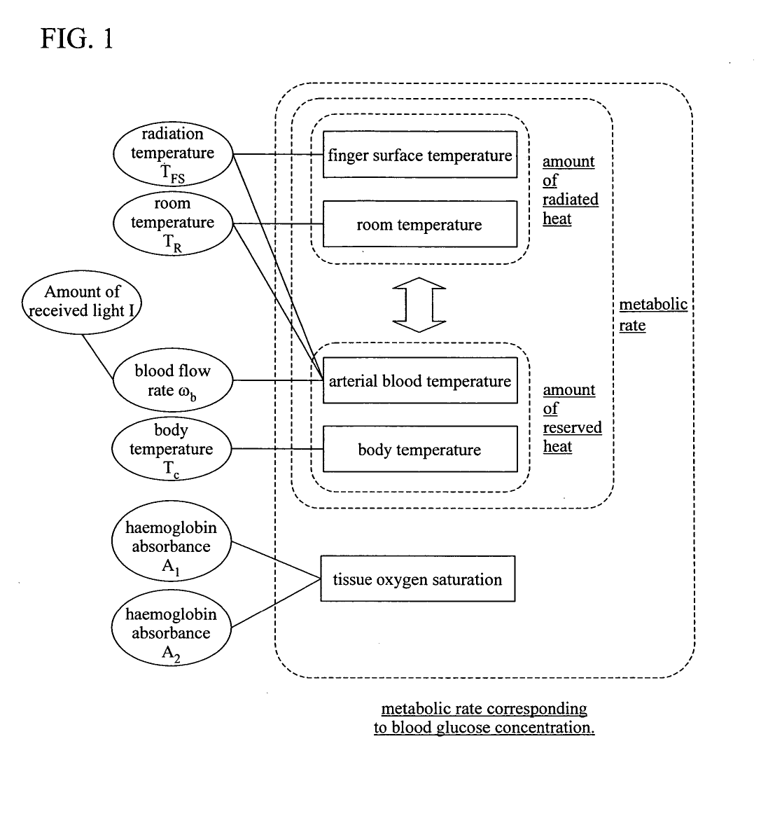

Now referring to FIG. 1, which shows a schematic block diagram illustrating the relationship between the measurements of various sensors and the parameters derived therefrom. Now the finger surface temperature T_{FS} is measured using a radiation thermometer and the room temperature T_{R} is measured with a thermistor. A thermometer connected to the apparatus or one commercially available is used for the measurement of body temperature Tc. The blood flow rate ω_{b} is calculated by measuring the photointensity I at the wavelength in relation to the haemoglobin absorbance. Parameters associated with the arterial blood temperature are derived from the finger body temperature TFS and the blood flow rate ω_{b}. Parameters associated with the haemoglobin oxygen saturation are derived from the measurement using absorbance A₁ and A₂ of at last two different wavelengths in relation to the haemoglobin absorbance.

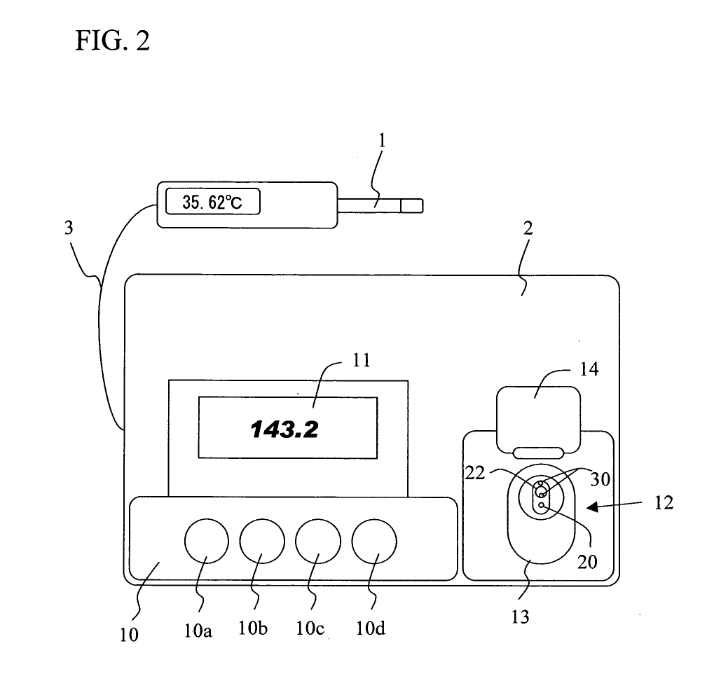

Now referring to FIG. 2, which is a top plan view of a metabolimeter according to the present invention. This apparatus uses the finger cushion skin as the body surface, however any other part of the body may be used instead, according to the measurement principle.

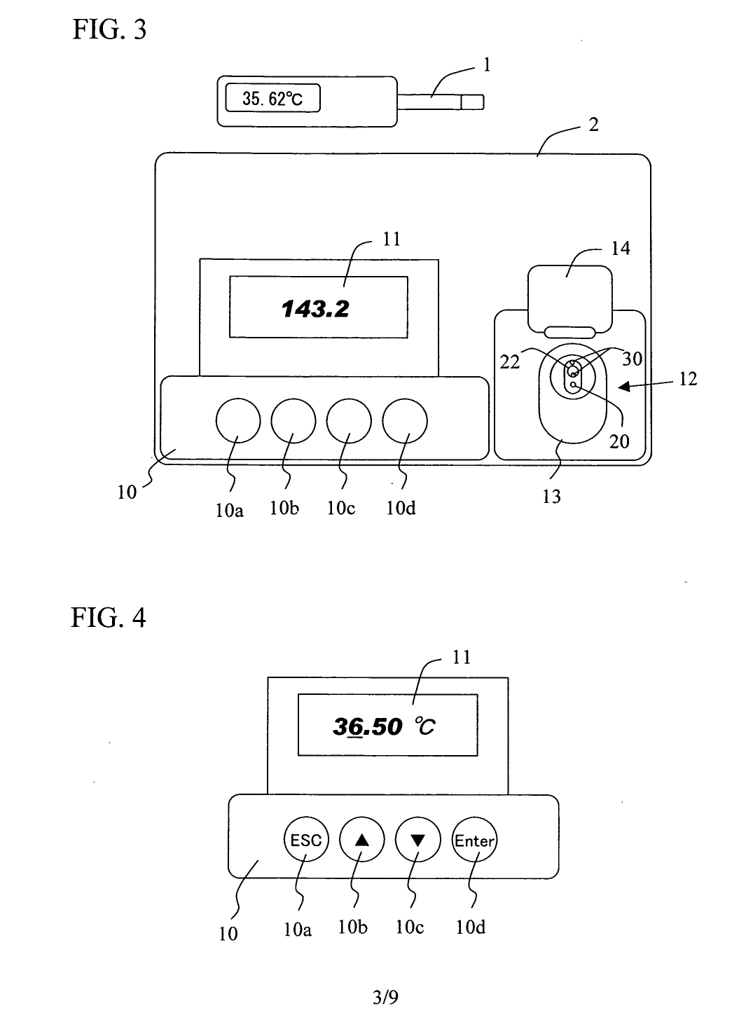

The apparatus is composed of a body temperature measuring unit 1 for measuring body temperature, and a main body 2 for measuring the room temperature, finger surface temperature, blood flow rate, and tissue oxygen saturation. The body temperature measuring unit 1 and the main body 2 are connected with a wiring 3. In the present embodiment, the body temperature measuring unit 1 is connected to the main body 2, and the body temperature data measured by the body temperature measuring unit 1 is transferred to the main body 2. However using a commercially available thermometer in common may be used to input the body temperature data via the operation unit 10 of the main body 2. FIG. 3 shows a top plan view of the metabolimeter according to the present invention with the body temperature and blood flow being measured with a generic device.

The body temperature data can be input by way of an example through a display screen as shown in FIG. 4. In this example 36.50%% DEGREE is displayed as the reference, the ones place is underscored to indicate that this digit is to be input. In this state buttons 10b and 10c are used to change the number. More specifically, button 10b is pushed to increase the displayed value while the button 10c is pushed to decrease the value so as for the input to be equal to the measured body temperature, then the button 10d is used to enter the ones place. Thereafter the numerical input is continued in a similar manner for the one and two decimal places to complete the body temperature data input. When the button 10d is pushed by error, button 10a is pushed to undo the input.

On the top of the apparatus an operation unit 10, a measuring unit 12 on which a finger subject to be measured is placed, a display unit 11 for displaying the measurement result, apparatus status and the measurements. Four push buttons 10a to 10d are arranged in the operation unit 10 for operating the apparatus. A cover 14 is attached on the measuring unit 12, a finger pad unit 13, having an ellipsoid circumference is under the cover 14 (the cover is opened in the figure). In the finger pad unit 13 an open end 22 of the radiation thermometric sensor unit, optical sensor unit 30, and temperature sensor unit 20 are provided.

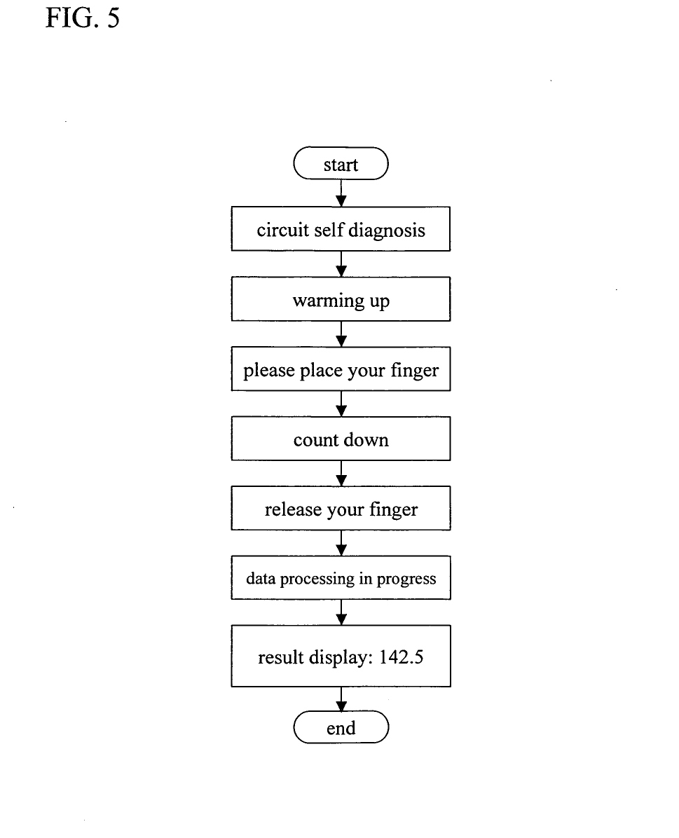

Now referring to FIG. 5, which shows the operation procedure of the apparatus. By pushing the button on the operation unit 10 to power on the apparatus, “warming up” is displayed on the display unit 11 and the electronics in the apparatus is warmed up. At the same time a checking program is loaded to automatically check the electronics. When “warming up” sign is over, an instruction “please place your finger” is displayed on the display unit 11. Once a finger is placed on the finger pad unit 13, the display unit 11 displays a down count. When the count down is complete, the display unit 11 indicates another instruction “release your finger”. Once the finger is released from the finger pad unit 13, the display unit 11 displays a message “data processing in progress”. Shortly after, the display unit 11 displays a metabolic rate corresponding to the blood glucose concentration. At this point, the displayed glucose level is stored in an IC card along with the date and time. After reading out the metabolic rate displayed, pushing the button on the operation unit causes the apparatus to cycle, and a message “please place your finger” on the display unit 11 for waiting next measurement appears in one minute.

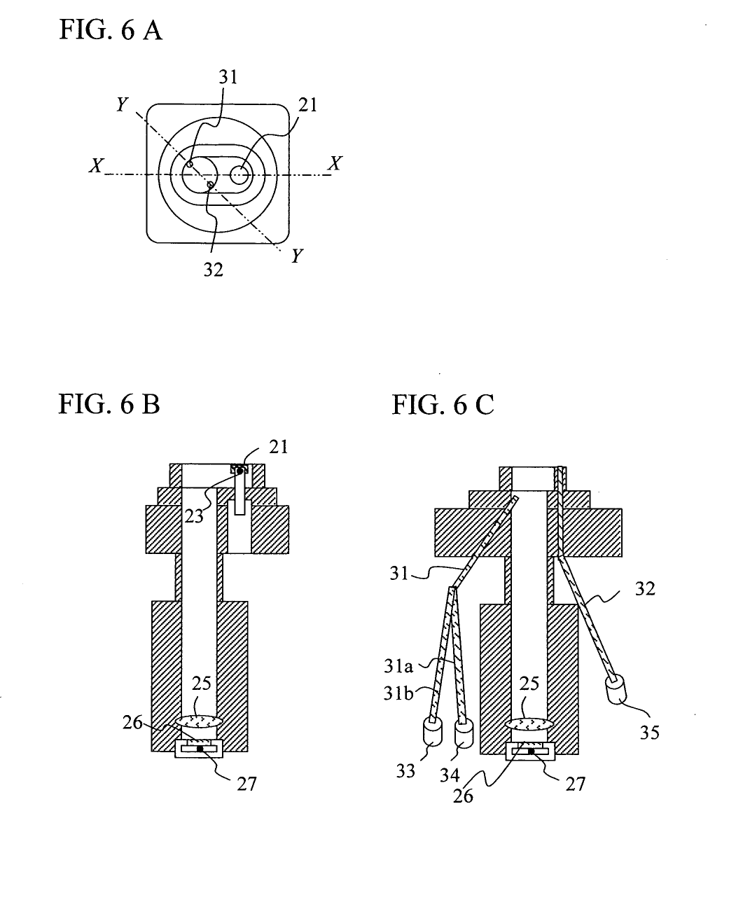

Now referring to FIGS. 6A to 6C, which shows the details of the measurement unit; FIG. 6A is a top plan view, FIG. 6B is a cross-sectional view taken along with the line X-X, and FIG. 6C is another cross-sectional view taken along with the line Y-Y.

Now the temperature measurement with the metabolimeter according to the present invention will be described. An infrared lens 25 is located in a position within the apparatus to see through the subject (finger cushion) placed on the finger pad unit 13. Beneath the infrared lens 25 a pyroelectric detector 27 is placed with an infrared transmissible window 26 therebetween. A thermistor 23 is used for measuring the room temperature, to define the temperature immediately prior to placing a finger as the room temperature.

The temperature sensor unit of the preferred embodiment has three temperature sensors, for measuring three different temperature values as follows—(1) radiation temperature of the finger (pyroelectric detector 27): T_{FS} (2) room temperature (thermistor 23): T_{R} and (3) body temperature (body temperature measuring unit 1): Tc

Next the optical sensor unit 30 will be described in greater details. The optical sensor unit measures the haemoglobin concentration and the haemoglobin oxygen saturation needed for determining the tissue oxygen saturation. The measurement of the haemoglobin concentration and haemoglobin oxygen saturation requires the optical density measurement with at least two different wavelengths. FIG. 6C shows an exemplary arrangement for two wavelength measurements by means of two light sources 33 and 34, and one single detector 35.

The optical sensor unit 30 houses the ends of two optical fibers 31 and 32. The optical fiber 31 is an optical fiber for light emission, and the optical fiber 32 is for receiving lights. As shown in FIG. 6C, the optical fiber 31 is connected to two optical fiber branches 31a and 31b, at the end of which two light emission diodes (LED) for two different wavelengths are mounted. At the end of the photoreception optical fiber 32 a photodiode 35 is provided. The LED 33 emits the light of wavelength 830 nm, while the LED 34 emits the light of wavelength 780 nm. These two near-infrared lights serve for the measurement of concentration variation of oxygenated and reduced haemoglobin as well as the measurement of total haemoglobin concentration variation, which corresponds to the sum of two state haemoglobin and to the volume of blood.

Two LEDs 33 and 34 emit light in a time-sharing manner, and the light beam emitted from those LEDs 33 and 34 is transmitted through the light emission optical fiber 31 to the finger of the subject. The light emitted to the finger is diffused and reflected by the finger skin and then is incident to photoreception optic fiber 32 to be detected by the detector 35. At the time when the light emitted to the finger is diffused and reflected by the finger skin, the light passes through the skin and penetrates into the tissue so that it is absorbed by the haemoglobin in the blood stream flowing through the capillaries. The measurement data from the detector 35 indicates the reflectance R, from which the absorbance may be approximately calculated as log (1/R). By emitting light of wavelength 830 nm and that of 780 nm, measuring the reflectance R for both wavelengths, as well as calculating log (1/R), the absorbance A1 for wavelength 830 nm and the absorbance A2 for wavelength 780 nm are both determined.

When defining the reduced haemoglobin concentration as [Hb], oxygenated haemoglobin concentration as [HbO₂], the absorbance A₁ and A₂ may be given by the following equation:

$$\begin{matrix}{A}_{1} = a \times ( [ Hb ] \times {A}_{Hb} ( 830 nm ) + [ {Hb O}_{2} ] \times {A}_{{Hb O}_{2}} ( 830 nm ) ) \\ = a \times ( [ Hb ] + [ {Hb O}_{2} ] ) \times {A}_{{HbO}_{2}} ( 830 nm )\end{matrix} \\ \begin{matrix}{A}_{2} = a \times ( [ Hb ] \times {A}_{Hb} ( 780 nm ) + [ {Hb O}_{2} ] \times {A}_{{Hb O}_{2}} ( 780 nm ) ) \\ = a \times ( [ Hb ] + [ {Hb O}_{2} ] ) \times ( \begin{matrix}( 1 - \frac{[ {Hb O}_{2} ]}{[ Hb ] + [ {Hb O}_{2} ]} ) \times {A}_{Hb} ( 780 nm ) + \\ \frac{[ {Hb O}_{2} ]}{[ Hb ] + [ {HbO}_{2} ]} \times {A}_{{Hb O}_{2}} ( 780 nm )\end{matrix} )\end{matrix} \qquad ( 16 )$$

A_{Hb}(830 nm) and A_{Hb}(780 nm), A_{HbO2}(830 nm) and A_{HbO2}(780 nm) are molar absorbance coefficient of reduced haemoglobin, and oxygenated haemoglobin, respectively, and are known in the art. a indicates a proportional index. Haemoglobin concentration [Hb]+[HbO₂], Haemoglobin oxygen saturation [HbO₂]/([Hb]+[HbO₂]) may be given from the equation above as follows:

$$[ Hb ] + [ {Hb O}_{2} ] = \frac{{A}_{1}}{a \times {A}_{{Hb O}_{2}} ( 830 nm )} \\ \frac{[ {Hb O}_{2} ]}{[ Hb ] + [ {Hb O}_{2} ]} = \frac{{A}_{2} \times {A}_{{Hb O}_{2}} ( 830 nm ) - {A}_{1} \times {A}_{Hb} ( 780 nm ) )}{{A}_{1} \times ( {A}_{{Hb O}_{2}} ( 780 nm ) - {A}_{Hb} ( 780 nm ) )} \qquad ( 17 )$$

It should be understood by those skilled in the art that although in this embodiment the absorbance measurement by using two wavelengths for determining the haemoglobin concentration and haemoglobin oxygen saturation has been described, absorbance measurement by using three or more wavelengths is also applicable, and may decreases the affection of interference to increase thereby the measurement precision.

The blood flow rate may be given from the frequency spectrum of the 780 nm light originated from the light emission diode 34 and diffused and reflected by the finger, as follows:
Tissue blood flow rate=k₁∫ωP(ω)dω/I² (18)

Where k₁ designates to the proportional index, ω to the angular frequency, P(ω) to the signal power spectrum, I to the amount of light received.

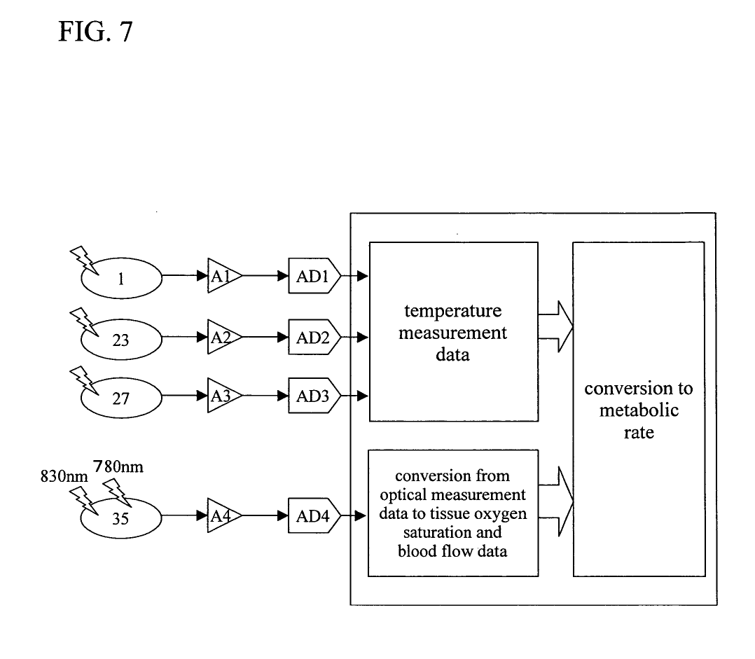

Now referring to FIG. 7, which shows diagrammatically a schematic block diagram illustrating the flow of data processing in the apparatus. The apparatus according to the preferred embodiment equips four sensor units each of which incorporates the body temperature measuring unit 1, a thermistor 23, a pyroelectric detector 27, and a photodiode 35. The photodiode 35 measures the absorbance of wavelength 830 nm and that of wavelength 780 nm. Five measurements are therefore input into the apparatus.

These five analog signals pass through respective amplifiers A1 to A4, then digitized by the A/D converters AD1 to AD4. From digitized values five physiological parameters (body temperature, room temperature, finger surface temperature, blood flow rate, tissue oxygen saturation) are calculated.

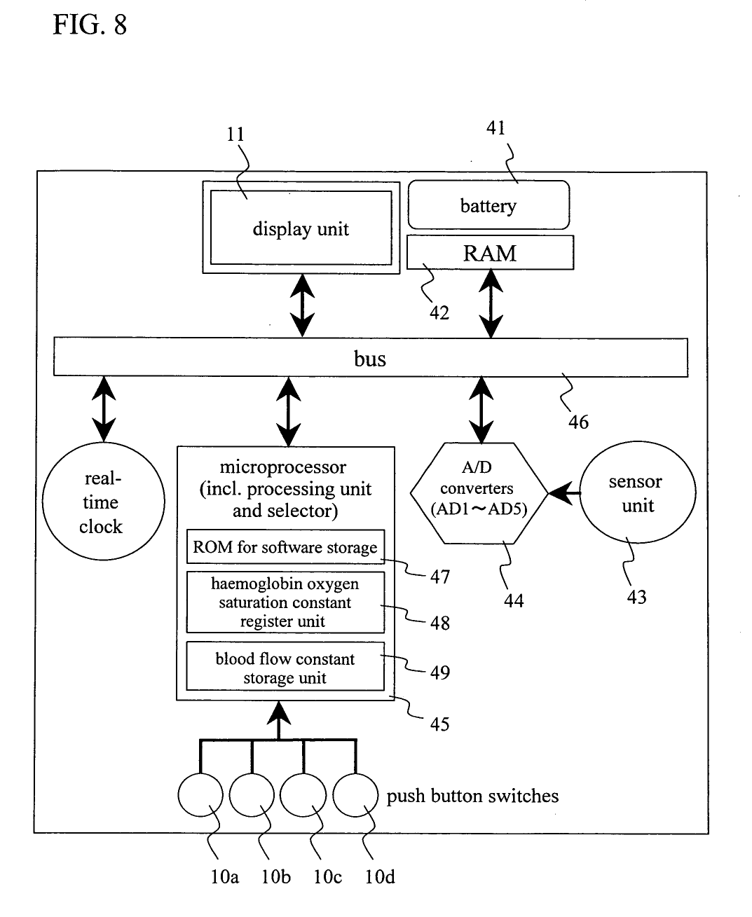

The conversion calculation is conducted from these five physiological parameters to the metabolic rate to be ultimately displayed. Now referring to FIG. 8, which shows the functional block diagram of the apparatus. The apparatus is driven by a battery 41. The signals measured by a sensor unit 43 including a temperature sensor and an optical sensor are fed to A/D converters 44 each arranged in correspondence with the respective signal (A/D converter AD1 to AD4) to be converted to digital signals. There are peripherals of a microprocessor 45, including A/D converters AD1 to AD4, LCD display unit 11, RAM 42, which are accessed by the microprocessor 45 via a bus 46, respectively. Push buttons 10a to 10d are each connected to the microprocessor 45. The microprocessor 45 receives external instructions by pushing the buttons 10a to 10d.

A ROM 47 incorporated in the microprocessor 45 stores any programs necessary for the processing. The microprocessor 45 also includes a haemoglobin oxygen saturation constant register unit 48 for storing the constant for the haemoglobin oxygen saturation, and a blood flow rate constant register unit 29 for storing the constant for the blood flow rate. The calculation program, after having performed the measurement of the finger, calls the optimum constants from the Hb/O₂ constant register unit and the blood flow constant register unit to start calculation. The memory area required for the processing is allocated in the RAM 42, which is also incorporated in the apparatus. The calculation result will be displayed on the display unit 11.

In addition, although the apparatus calculates the metabolic rate therein in the present embodiment, the physiological parameters may also be stored in an IC card, and another PC reads the data on the IC card to calculate the metabolic rate.

The ROM stores any program components necessary for the processing, especially function for determining the metabolic rate. The function is defined as follows. First, the metabolic rate is expressed as equation (9). The indexes a to e in equation (9) are predetermined based on a plurality of measurements. The procedure for determining the a to e (aᵢ, (i=0, 1, 2, 3, 4)) is as follows.

(1) prepare physiological parameters and multiple regression indicative of the metabolic rate of another measurement.

(2) determine a normal equation (simultaneous equation) with respect to the physiological parameters from the equation obtained by the least square method.

(3) determine the indexes aᵢ (i=0, 1, 2, 3, 4) from the normal equation and assign the value into the multiple regression.

Glucose concentration is expressed as equation (15). The indexes a to f in equation (15) are predetermined based on a plurality of measurements. The procedure to determine a to f (aᵢ, (i=0, 1, 2, 3, 4, 5) may be the same as that of the metabolic rate. Now an exemplary case of glucose concentration is shown here. First, the following regression is created, which indicates the relationship between the blood glucose concentration Gc and physiological parameters X₁=(T_{FS}−T_{R})/ω_{b}, X₂=T_{FS}/ω_{b}, X₃=(T_{FS}−T_{R}), X₄=Tc, X₅=StO₂.

$$\begin{matrix}C = f ( {X}_{1} , {X}_{2} , {X}_{3} , {X}_{4} , {X}_{5} ) \\ = {a}_{0} + {a}_{1} {X}_{1} + {a}_{2} {X}_{2} + {a}_{3} {X}_{3} + {a}_{4} {X}_{4} + {a}_{5} {X}_{5}\end{matrix} \qquad ( 19 )$$

Next, the least square method is used to define a multiple regression so as to minimize the error with the blood glucose concentration measurements Gc measured by an enzymatic electrode method. By defining the sum of squares of the residual error as D, D may be given by the following equation:

$$\begin{matrix}D = {\sum }_{i = 1}^{n} {d}_{i}^{2} \\ = {\sum }_{i = 1}^{n} {( {C}_{i} - f ( {X}_{i 1} , {X}_{i 2} , {X}_{i 3} , {X}_{i 4} , {X}_{i 5} ) )}^{2} \\ = {\sum }_{i = 1}^{n} {\{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \}}^{2}\end{matrix} \qquad ( 20 )$$

Since where the sum of squares of residual becomes minimum is when equation (20) partially differentiated by a₀, a₁, . . . , a₅ becomes 0, the following equation can be given:

$$\frac{\partial D}{\partial {a}_{0}} = - 2 {\sum }_{i = 1}^{n} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial D}{\partial {a}_{1}} = - 2 {\sum }_{i = 1}^{n} {X}_{i 1} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial D}{\partial {a}_{2}} = - 2 {\sum }_{i = 1}^{n} {X}_{i 2} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial D}{\partial {a}_{3}} = - 2 {\sum }_{i = 1}^{n} {X}_{i 3} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial D}{\partial {a}_{4}} = - 2 {\sum }_{i = 1}^{n} {X}_{i 4} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \\ \frac{\partial D}{\partial {a}_{5}} = - 2 {\sum }_{i = 1}^{n} {X}_{i 5} \{ {C}_{i} - ( {a}_{0} + {a}_{1} {X}_{i 1} + {a}_{2} {X}_{i 2} + {a}_{3} {X}_{i 3} + {a}_{4} {X}_{i 4} + {a}_{5} {X}_{i 5} ) \} = 0 \qquad ( 21 )$$

Now defining mean of C, X₁ to X₅ as Cₘₑₐₙ, X₁ₘₑₐₙ to X₅ₘₑₐₙ, then Xᵢₘₑₐₙ=0 (i=1-5), the following equation (22) may be given from equation (19):

$$\begin{matrix}{a}_{0} = {C}_{mean} - {a}_{1} {X}_{1 mean} - {a}_{2} {X}_{2 mean} - \\ {a}_{3} {X}_{3 mean} - {a}_{4} {X}_{4 mean} - {a}_{5} {X}_{5 mean} \\ = {C}_{mean}\end{matrix} \qquad ( 22 )$$

Variation and covariation between normalized parameters are expressed by equation (23), and covariation between normalized parameters Xᵢ (i=1-5) and C are expressed by equation (24):

$$\begin{matrix}{S}_{ij} = {\sum }_{k = 1}^{n} ( {X}_{ki} - {X}_{imean} ) ( {X}_{kj} - {X}_{jmean} ) = {\sum }_{k = 1}^{n} {X}_{ki} {X}_{kj} ( i , j = 1 , 2 , … 5 ) & ( 23 ) \\ {S}_{iC} = {\sum }_{k = 1}^{n} ( {X}_{ki} - {X}_{imean} ) ( {C}_{k} - {C}_{mean} ) = {\sum }_{k = 1}^{n} {X}_{ki} ( {C}_{k} - {C}_{mean} ) ( i = 1 , 2 , … 5 ) & ( 24 )\end{matrix}$$

Now assigning equations (22), (23), and (24) into equation (21) to digest to yield a simultaneous equation (normal equation) (25). By solving equation (25) a₁-a₅ may be given.
a₁S₁₁+a₂S₁₂+a₃S₁₃+a₄S₁₄+a₅S₁₅=S_{1C}
a₁S₂₁+a₂S₂₂+a₃S₂₃+a₄S₂₄+a₅S₂₅=S_{2C}
a₁S₃₁+a₂S₃₂+a₃S₃₃+a₄S₃₄+a₅S₃₅=S_{3C}
a₁S₄₁+a₂S₄₂+a₃S₄₃+a₄S₄₄+a₅S₄₅=S_{4C}
a₁S₅₁+a₂S₅₂+a₃S₅₃+a₄S₅₄+a₅S₅₅=S_{5C} (25)

Constant term a₀ may be given by equation (22). The aᵢ (i=0, 1, 2, 3, 4, 5) as defined as above is stored on the ROM as manufacturer supplied. In the actual measurement using the apparatus, normalized parameters Xᵢ to X₅ determined from the measurements are substituted for the regression equation (19) to calculate the glucose concentration. It should be noted here that the aᵢ (i=0, 1, 2, 3, 4, 5) may be defined for the user after delivery to store in the IC card, instead of as-manufactured basis.

A specific example of calculation of metabolic rate will be described in greater details herein below. The indexes of equation (9) are defined from the data of many measurements performed in advance on a number of persons, and the ROM of the microprocessor stores the following equation of metabolic rate.
Metabolic rate=13415−308T_{c}−31(T_{FS}−T_{R})+38(T_{FS}−T_{R})/ω_{b}−6.4T_{FS}/ω_{b}

As an example of measurements, the equation above is substituted with measurement data Tc=36.45, T_{FS}−T_{R}=7.80, (T_{FS}−T_{R})/ω_{b}=13.11, T_{FS}/ω_{b}=54.03 to yield 8.8 kJ. The metabolic rate measured by an indirect calorimetric measurement method in a closed circuit of the same is 8.7 kJ. The measurement data when the metabolic rate is obtained as 8.2 kJ in the indirect calorimetric measurement method in a closed circuit, i.e., Tc=36.99, T_{FS}−T_{R}=6.76, (T_{FS}−T_{R})/ω_{b}=7.68, T_{FS}/ω_{b}=35.41 may be substituted for the equation to obtain 7.9 kJ.

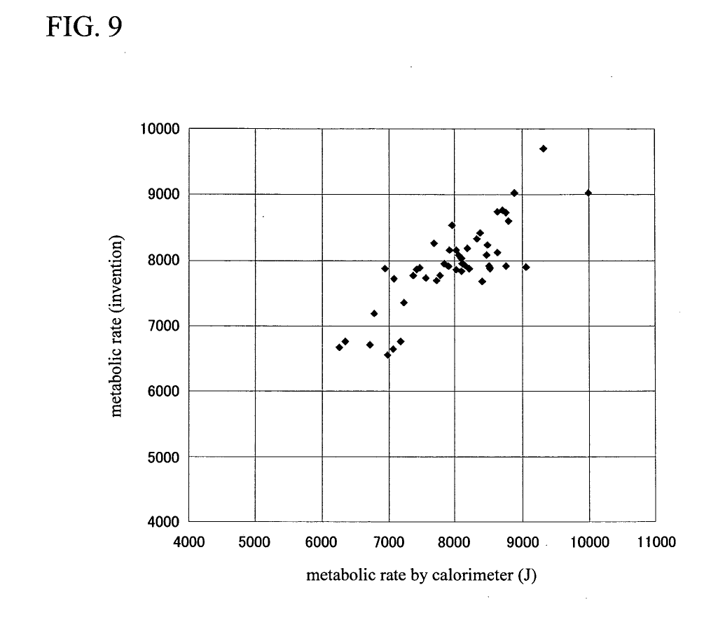

Now referring to FIG. 9, which shows a plot of measurements obtained from a plurality of subjects, with the ordinate plotting the calculated values of metabolic rate according to the present invention, and the abscissa plotting the measurements of metabolic rate by means of the indirect calorimetric measurement method in a closed circuit system. In the inventive method a better correlation is obtainable by calculating the metabolic rate from the measurement of human body temperature, finger temperature, and blood flow rate (correlative index=0.82).

A specific example of calculation of glucose concentration will be described in greater details herein below. The index of equation (15) is defined from the data of many measurements performed in advance on a number of people, and the following calculation equation of glucose concentration is stored in the ROM of the microprocessor.
Gc=2256.8−54.4T_{c}+8.7(T_{FS}−T_{R})−4.95(T_{FS}−T_{R})/ω_{b}−0.7T_{FS}/ω_{b}−182.3StO₂

As a typical example of measurements, substituting measurement data Tc=36.45, T_{FS}−T_{R}=7.80, (T_{FS}−T_{R})/ω_{b}=13.11, T_{FS}/ω_{b}=54.03, StO₂=0.72 for the equation above may yield 148.1. The blood glucose concentration at that time is 178.6 mg/dl. Then substituting the measurement data at the time of blood glucose concentration 203.4 mg/dl, Tc=36.52, T_{FS}−T_{R}=7.81, (T_{FS}−T_{R})/ω_{b}=11.30, T_{FS}/ω_{b}=46.51, StO₂=0.49 for the above equation yields 201.5 mg/dl.

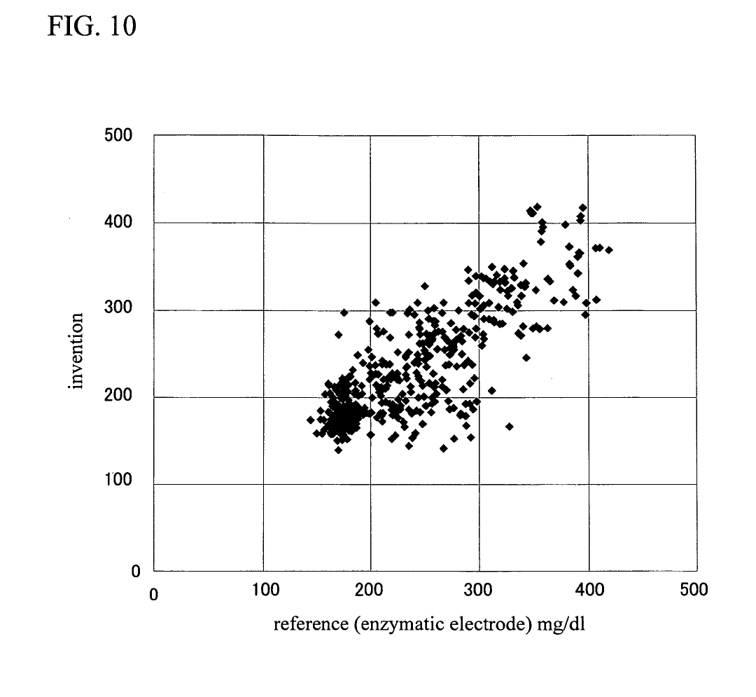

Now referring to FIG. 10, which shows a plot of measurements obtained from a plurality of subjects, with the ordinate plotting the calculated values of glucose concentration according to the present invention, and the abscissa plotting the measurements of glucose concentration by means of an enzymatic electrode method. In the inventive method a better correlation is obtainable by calculating the blood glucose concentration from the measurements of human body temperature, finger temperature, volume of supplied oxygen, and blood flow rate (correlative index=0.84).

The metabolic rate can be calculated which corresponds to the blood glucose concentration by multiplying the calculated glucose concentration by the index A of equation (11).

It should be noted here that although the metabolic rate is investigated which correspond to the glucose concentration in the preferred embodiment, the metabolic rate may equally be based on the neutral fat (acylglycerol) and/or cholesterol as the metabolic rate of a substance other than the glucose, the equations may be defined from the blood concentration and measurements to determine the metabolic rate corresponding to the neutral fat and cholesterol concentration.

With respect to the calculated metabolic rate, or glucose concentration, or metabolic rate corresponding to the blood glucose concentration, the measurements accumulated from the group of healthy adults has the tendency of being different from those accumulated from the group of patients of such metabolic diseases as arteriosclerosis, heart diseases, glucose tolerance anomaly and the like. The calculated values may be clues as to the underlying diseases. The time-series display of accumulated metabolic rate allows facilitating the diagnosis of the tendency.

The present embodiment has the identical configuration as the preceding embodiment, the similar members are designated to the identical reference numbers and the detailed description of the parts already described in the preceding embodiment will be omitted.

The present invention may be embodied in other specific forms without departing from the spirit or essential characteristics thereof. For instance, (effect of the invention)

It is to be understood that the present invention is not to be limited to the details herein given but may be modified within the scope of the appended claims.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

It is further understood by those skilled in the art that the foregoing description is a preferred embodiment of the disclosed device and that various changes and modifications may be made in the invention without departing from the spirit and scope thereof.

What is claimed is:

1. A metabolic rate measurement apparatus, comprising:
an environmental thermometric sensor unit for measuring environmental temperature;
a first position thermometric sensor unit for measuring temperature derived from a first position on body surface of a subject,
a second position temperature acquiring mean acquiring temperature of a second position, the second position being different from said first position at the subject;
a blood flow information acquiring mean acquiring information about the blood flow at said first position;
a storage unit for storing the environment temperature, the temperature derived from said first position, the temperature of said second position, and relationship between metabolic rate and the blood flow;
a processing unit for computing the metabolic rate by applying the environment temperature measured by said environment thermometric sensor unit, first position temperature measured by said first position thermometric sensor unit, second position temperature acquired by said second position temperature acquiring mean, and the blood flow information acquired by said blood flow information acquiring mean to the relationship stored in said storage unit; and
a display unit for displaying the computational result from said processing unit.

2. A metabolic rate measurement apparatus, according to claim 1, wherein:
said second position temperature acquiring mean is comprised of a body thermometer, the output of said body thermometer being supplied to said processing unit.

3. A metabolic rate measurement apparatus, according to claim 1, wherein:
said second position temperature acquiring mean has an operation unit for inputting body temperature as numeric data.

4. A metabolic rate measurement apparatus, according to claim 1, wherein:
said blood flow information acquiring mean has a light source for emitting light to said first position, and a photometer for measuring the frequency spectrum of light diffused and reflected from said first position.

5. A metabolic rate measurement apparatus, according to claim 1, wherein:
said blood flow information acquiring mean has an operation unit for inputting the blood flow rate as numeric data.

6. A metabolic rate measurement apparatus, according to claim 1, further comprising:
an oxygen saturation information acquiring mean acquiring information about the oxygen saturation at said first position,
wherein
said storage unit stores the temperature derived from said first position, the temperature of said second position, and the relationship between said blood flow rate and said oxygen saturation and the metabolic rate;
said processor unit calculates metabolic rate by applying the environment temperature measured by said environment temperature sensor unit, the first position temperature measured by said first position thermometric sensor unit, the second position temperature measured by said second position temperature acquiring mean, the blood flow information acquired by said blood flow information acquiring mean , and the oxygen saturation acquired by said oxygen saturation information acquiring mean to said relationship stored in said storage unit.

7. A metabolic rate measurement apparatus, according to claim 6, wherein:
said oxygen saturation information acquiring mean has a light source for emitting light of at least two different wavelengths, a detector for detecting light originated from said light source and diffused by said body surface, and a means for calculating haemoglobin oxygen saturation based on detection results from said detector unit.

8. A metabolic rate measurement apparatus, according to claim 6, wherein:
said oxygen saturation information acquiring mean has an operation unit for entering the oxygen saturation as numerical data.

9. A metabolic rate measurement apparatus, according to claim 1, wherein:
said first position thermometric sensor unit has a pad unit for placing a finger, and a temperature sensor for measuring the temperature derived from said first position of the finger placed on said pad unit.

10. A metabolic rate measurement apparatus, according to claim 9, wherein:
said temperature sensor measures radiation temperature derived from said first position.

11. A metabolic rate measurement apparatus, comprising:
an environment temperature sensor unit for measuring the environmental temperature;
a body surface contact unit for contacting with a first position of the body surface,
a cylinder member, contacted to said body surface contact unit and having an opening at one end;
a radiation thermometric sensor mounted in the vicinity of the other end of said cylinder member, for measuring radiation heat from said first position;
a blood flow information acquiring mean acquiring information about the blood flow at said first position;
a light source for emitting light of at least two different wavelengths into the one end of said cylinder member,
a light sensor unit for detecting light interacted with said body surface;
a second position temperature acquiring mean acquiring temperature of a second position, the second position being different from said first position at said body surface;
a processing unit for computing the metabolic rate by applying the environment temperature measured by said environment thermometric sensor unit, temperature of first position measured by said radiation thermometric sensor unit, temperature of second position acquired by said second position temperature acquiring mean, and blood flow rate information acquired by said blood flow information acquiring mean, and light detection result detected by said light detector unit to previously stored equation of the metabolic rate; and
a display unit for displaying output from said processing unit.

12. A metabolic rate measurement apparatus, according to claim 11, wherein:
said second position temperature acquiring meanis a body thermometer.

13. A metabolic rate measurement apparatus, according to claim 11, wherein:
said second position temperature acquiring meanis an operation pad for entering the body temperature as numeric data.

14. A metabolic rate measurement apparatus, according to claim 11, wherein:
said first position is to be a part in one of four limbs of the subject to be measured, and said radiation heat detector unit measures the radiation heat derived from said part.

15. A metabolic rate measurement apparatus, according to claim 11, further comprising a data storage unit for storing output from said processor unit, wherein:
data stored in said data storage unit is displayed on said display unit in a time-series manner.

16. A metabolic rate measurement apparatus, according to claim 11, wherein:
said metabolic rate is the metabolic rate of glucose.

17. A metabolic rate measurement apparatus, according to claim 11, wherein:
said metabolic rate is the metabolic rate of neutral fat and/or cholesterol.
